(19) United States
(12) Patent Application Publication
(10) Pub. No.: US 2006/0041296 A1
(43) Pub. Date: Feb. 23, 2006

(54) Novel medical electrode configurations

(75) Inventors: Ryan T. Bauer, Brooklyn Park, MN (US); Kiem H. Dang, Blaine, MN (US); John L. Sommer, Coon Rapids, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/924,024

(22) Filed: Aug. 23, 2004

(51) Int. Cl. A61N 1/05

(52) U.S. Cl. 607/122

(57) ABSTRACT

A medical electrode includes an active surface extending from a first end to a second end and has a maximum outer diameter exceeding an outer diameter of the both the first end and the second end. The electrode further includes a recess formed in the active surface and an agent held in the recess and adapted to disperse out from the recess upon implantation of the electrode.

### TECHNICAL FIELD

The present invention pertains to medical electrical systems and more particularly to electrode assemblies.

### BACKGROUND

Cardiac stimulation systems commonly include a pulse-generating device, such as a pacemaker or implantable cardioverter/defibrillator that is electrically connected to the heart by at least one medical electrical electrode. A medical electrical electrode delivers electrical pulses emitted by the device to the heart and may also sense cardiac signals so the device may monitor the electrical activity of the heart. These electrical pulses are typically conducted between the device and electrodes via elongate conductors extending within one or more leads.

In recent years, with the development of cardiac resynchronization therapy, pacing of the left ventricle has been achieved by implanting transvenous lead electrodes in vessels of the coronary venous system of the heart in order to stimulate an epicardial surface of the left ventricle. Thus there is a need for electrode assemblies that are suited for delivery to, and function within in a vessel environment.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1A is a plan view of a medical electrical lead according to one embodiment of the present invention;

FIG. 1B is a schematic of the lead of FIG. 1A implanted in a coronary venous system from an anterior perspective;

FIG. 1C is an enlarged view of a distal portion of the lead shown in FIG. 1A implanted within a coronary vein;

FIG. 2 is an enlarged detailed plan view of a lead electrode assembly according to one embodiment of the present invention; and

FIG. 3 is an enlarged detailed section view of another lead electrode assembly according to another embodiment of the present invention.

### DETAILED DESCRIPTION

The following detailed description is exemplary in nature and is not intended to limit the scope, applicability, or configuration of the invention in any way. Rather, the following description provides a practical illustration for implementing exemplary embodiments of the invention.

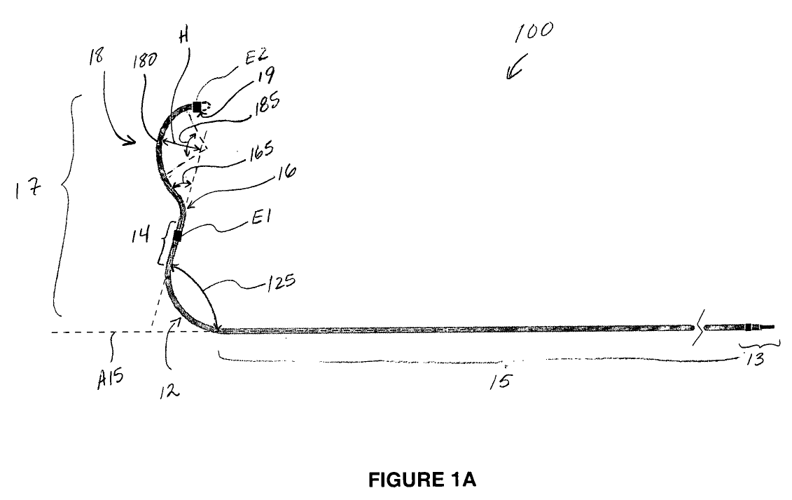

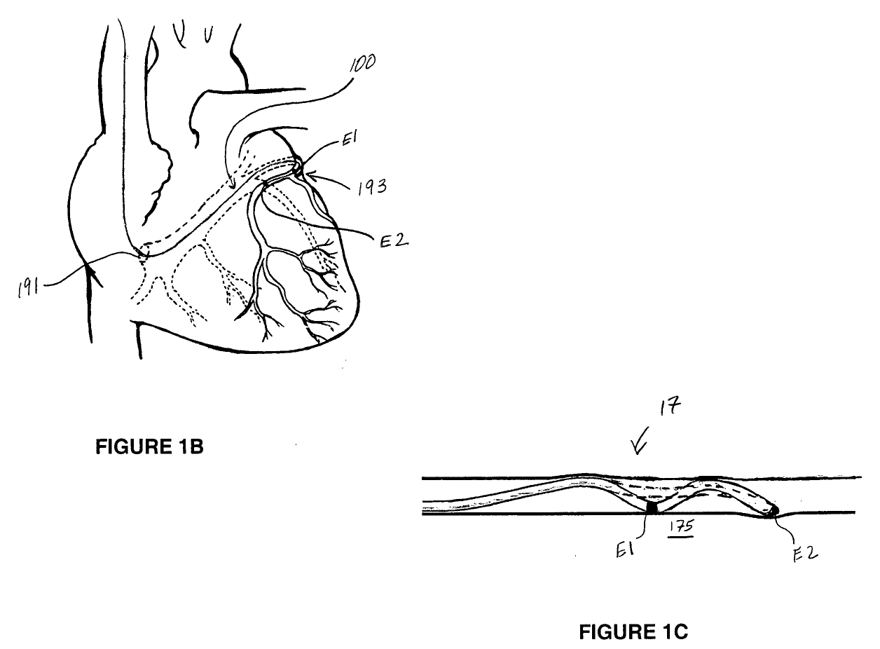

FIG. 1A is a plan view of a medical electrical lead 100 according to one embodiment of the present invention. FIG. 1A illustrates lead 100 including an approximately straight proximal lead body portion 15, which is terminated at a proximal end by a lead connector 13, and a pre-formed distal lead body portion 17 extending distally from proximal portion 15. FIG. 1A further illustrates distal lead body portion 17 including a first arcuate segment 12 bending in a first direction, an approximately straight segment 14 extending from first arcuate segment 12, a second arcuate segment 16 extending from straight segment 14 and bending in a second, generally distal, direction, a third arcuate segment 18 bending in a third, generally proximal, direction, and a distal tip segment 19 extending from the third arcuate segment 18. According to the illustrated embodiment of the present invention, lead 100 further includes a first electrode E1 coupled to approximately straight segment 14 and second electrode coupled to distal tip segment 19; the position of preformed curves of arcuate segments of distal portion 17 with respect to electrodes E1 and E2 provide for epicardial contact of electrodes E1 and E2 when implanted in a coronary vessel, as will be further described below.

FIG. 1A further illustrates angles 125, 165 and 185 of arcs included in arcuate segments 12, 16 and 18, respectively; according to some embodiments of the present invention, dimensions of the arcs are as indicated in Table 1.

TABLE 1
Arc Dimensions

| Arcuate Segment | Arc radius (inch) range | Arc angle range |
|---|---|---|
| 12 | ˜0.2-˜0.3 | Angle 125: ˜45°-˜90° |
| 16 | ˜0.2-˜0.4 | Angle 165: ˜10°-˜40° |
| 18 | ˜0.1-˜0.4 | Angle 185: ˜60°-˜100° |

Furthermore, a length of straight segment 14, according to some embodiments, is from approximately 0.2 to approximately 0.7 inch and a length of distal tip segment 19 is from approximately 0.05 inch to approximately 0.2 inch. According to one embodiment electrode E2 terminates distal tip segment 19, which may or may not extend proximally from electrode; according to another embodiment a portion of distal tip segment 19 extends distally from electrode E2 as illustrated by dashed lines in FIG. 1 and this extension may or may not be curved. Distal lead body portion 17 is alternately described as being canted, bending at angle 125 with respect to a longitudinal axis Al 5 of proximal portion 15 and including a hump-like segment, corresponding to segment 18, extending from approximately straight segment 14 and having a distal apex 180. According to one embodiment of the present invention, the arc of segment 18 has a chord length of approximately 0.4 inch to approximately 0.7 inch and distal apex 180 of segment 18 has a height H of approximately 0.1 inch to approximately 0.3 inch.

General construction details concerning lead 100, for example of arrangement of conductors and insulation, coupling of electrodes to conductors, and assembly of connector 13, are well known to those skilled in the art. Conductors coupling electrodes E1 and E2 to connector contacts of connector 13 may be side-by-side cables or coaxial coils, either of which may be formed of wires made from MP35N alloy; and insulation formed about conductors for electrical isolation may formed of polyurethane, fluoropolymers, silicone, polyimide or any combination thereof. Methods for pre-forming distal portion 17 include pre-forming of conductors extending therein and/or sheaths extending about the conductors; according to one method one or more sheaths extending between proximal lead body portion 15 and distal tip segment 17 are formed of polyurethane, which is heat set into the preformed curve; such a method is further described in U.S. Pat. No. 5,999,858, which is incorporated herein by reference.

FIG. 1B is a schematic of lead 100 implanted in a coronary venous system 193, and FIG. 1C is an enlarged view of distal lead body portion 17 therein. FIG. 1B illustrates lead 100 having been passed through a coronary sinus 191 into coronary vasculature 193 such that electrodes E1 and E2 are positioned for left ventricular pacing. According to some embodiments of the present invention both electrodes E1 and E2 are designed for pacing stimulation so that one of the two electrodes may be selected for ventricular pacing based on a preferred implant position; as illustrated in FIG. 1C, the pre-formed curvature of distal lead body portion 17 assures that both electrodes E1 and E2 contact a left ventricular epicardial surface 175. Electrodes E1 and E2 may each have a surface area ranging between approximately 2 square millimeters and approximately 10 square millimeters and may be formed from any suitable material known to those skilled in the art, for example platinum-iridium and titanium. Dashed lines in FIG. 1C show an alternate distal lead body portion wherein a pre-formed hump (i.e. segment 18, FIG. 1A) is not included in order to illustrate a need for the hump when two electrodes are included in the distal lead body portion. FIG. 1C also shows how canted distal portion 17 serves to force electrode E2 into contact with epicardial surface 175.

FIG. 1C further illustrates that pre-formed segments 12, 16 and 18 (FIG. 1A) of distal portion 17 are flexible to bend in compliance with external forces such as that applied by the vessel walls of coronary vasculature 193. These segments may also be bent in compliance with an internal force applied by a stylet inserted within a lumen of lead 100.

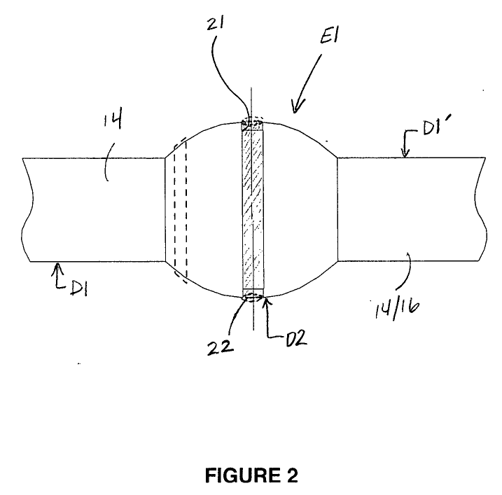

FIG. 2 is an enlarged detailed plan view of a lead electrode assembly, corresponding to first electrode E1 illustrated in FIGS. 1A-C, according to one embodiment of the present invention. FIG. 2 illustrates approximately straight segment 14 of distal lead body portion 17 extending away from electrode E1 toward segment 12(FIG. 1A); E1 may be positioned along segment 14 such that segment 14 further extends in an opposite direction from electrode E1, or such that electrode E1 is in close proximity or adjacent to second arcuate segment 16 (thus segment 14/16 indicated in FIG. 2). FIG. 2 further illustrates electrode E1 including a central portion having a maximum diameter D2 that is greater than diameters D1 and D1′ of segments 14 and 14/16, respectively, while either end of electrode E1 is approximately flush with diameters D1 and D1′. According to some embodiments of the present invention, a ratio of diameter D2 to diameters D1 and D1′ is from approximately 1.1 to approximately 1.6. It is likely that an active outer surface of electrode E1 in proximity to D2 will make best contact with epicardial tissue, for example epicardial surface 175 illustrated in FIG. 1C.

According to the illustrated embodiment the active outer surface of electrode E1 has a generally arcuate profile and includes a recess 21, approximately aligned with a longitudinal center of electrode E1 and in which a therapeutic or bioactive agent 22 is held, agent 22 being adapted to disperse out from recess 21 upon implantation of electrode E1. According to an alternate embodiment, a recess holding an agent is offset from the longitudinal center of E1, as illustrated in FIG. 2 with dashed lines in proximity to segment 14. Although FIG. 1 illustrates recess extending about a circumference of electrode E1, alternate embodiments of the present invention include recesses, of a generally macroscopic scale, which are discrete in nature and of various orientations. Other dashed lines in FIG. 2 illustrate alternate profiles of agent 22 including arcuate and flat profiles which may be either protruding, flush or recessed with respect to adjacent outer surface of electrode E1. According to one set of embodiments of the present invention, agent 22 is embedded in a polymer matrix, and, according to a particular embodiment, agent 22 is an anti-inflammatory agent such as a steroid, for example dexamethasone sodium phosphate, dexamethasone acetate, or beclomethasone diproprionate, embedded in a polyurethane or silicone matrix such that the steroid may elute from the matrix to prevent inflammation at the electrode contact site. Methods for forming such compounds for application in embodiments of the present invention are well known to those skilled in the art. According to another set of embodiments, a surface of recess 21 includes a microstructure in which agent 22 is embedded, for example a platinized surface in which beclomethasone is embedded.

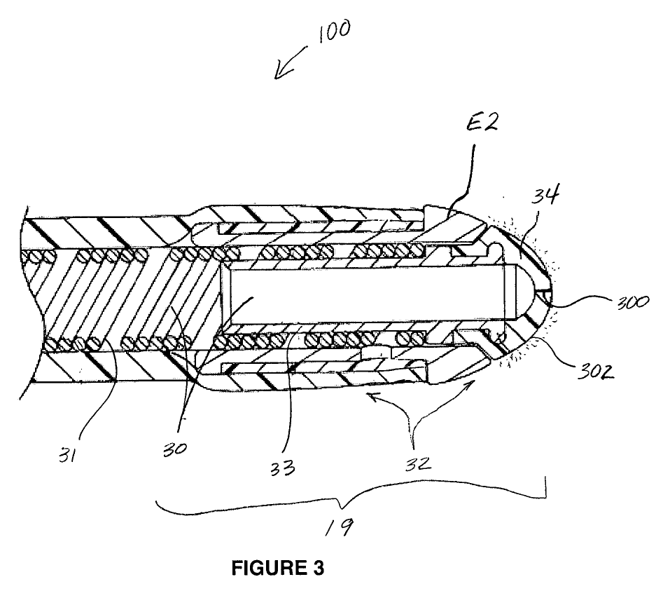

FIG. 3 is an enlarged detailed section view of another lead electrode assembly, corresponding to second electrode E2 illustrated in FIGS. 1A-C, according to another embodiment of the present invention. FIG. 3 illustrates lead 100 including a lumen 30 formed by a conductor coil 31 and a core 33 to which conductor coil 31 and electrode E2 are coupled; lumen 30 is terminated at a distal end of distal tip segment 19 with a resilient element 34 mounted upon core 33 and adjacent to electrode E2. According to the illustrated embodiment, element 34 is generally cup shaped and includes an outer surface 302, which forms a portion of an external surface 32 of distal tip segment 19 of distal lead body portion 17 (FIG. 1A), and an inner surface 300 adapted both to seal off lumen 30 and to spread apart to allow passage of an elongate member, for example a guide wire, by nature of the resiliency of element 34. U.S. Pat. No. 6,192,280 describes in part the assembly illustrated in FIG. 3 and is incorporated herein in its entirety. According to some embodiments of the present invention, element 34 further includes a therapeutic or bioactive agent embedded therein which is adapted to disperse out from outer surface 302 upon implantation of lead 100. According to one embodiment, the agent is an anti-inflammatory agent such as a steroid, for example dexamethasone sodium phosphate, dexamethasone acetate, or beclomethasone diproprionate, and element 34 is formed by transfer molding a blend of the steroid (10%-50% by weight) and a silicone rubber, according to methods known to those skilled in the art of silicone molding.

In the foregoing detailed description, the invention has been described with reference to specific embodiments. However, it may be appreciated that various modifications and changes can be made without departing from the scope of the invention as set forth in the appended claims. For example, the inventive electrode assemblies described herein are not limited to the lead body embodiments described herein and may be incorporated in many types of medical electrical systems. Furthermore, although embodiments of the present invention have been described herein in the context of cardiac pacing from the coronary venous vasculature, the scope of the present invention is not limited to this particular application and embodiments of the present invention may be applied to other vessel-like environments.

What is claimed is:

1. A medical electrical lead, comprising:
an elongate lead body including a distal portion, the distal portion including a first segment and a second segment, the first and second segments each having an outer diameter;
an electrode coupled to the lead body distal portion between the first and second segments and comprising:
a first end and a second end, the first end approximately flush with the first segment outer diameter and the second end approximately flush with the second segment outer diameter, and
an outer surface extending from the first end to the second end and including a recess formed therein and a maximum diameter, the maximum diameter exceeding the outer diameter of the first segment and the second segment; and
an agent held in the recess of the outer surface of the electrode, the agent adapted to disperse out from the recess upon implantation of the lead.

2. The lead of claim 1, wherein the outer surface of the electrode has a generally arcuate profile.

3. The lead of claim 1, wherein the maximum diameter of the outer surface of the electrode is approximately aligned with a longitudinal center of the electrode.

4. The lead of claim 1, wherein the recess extends around a circumference of the outer surface of the electrode.

5. The lead of claim 1, wherein the recess is offset from a longitudinal center of the electrode.

6. The lead of claim 1, wherein the recess is approximately aligned with a longitudinal center of the electrode.

7. The lead of claim 1, wherein the agent is an anti-inflammatory agent.

8. The lead of claim 1, further comprising a polymer matrix in which the agent is embedded.

9. The lead of claim 8, wherein the polymer matrix includes an outer surface protruding from a portion of the outer surface of the electrode, which is adjacent the recess.

10. The lead of claim 8, wherein the polymer matrix includes an outer surface recessed below a portion of the outer surface of the electrode, which is adjacent the recess of the outer surface.

11. The lead of claim 8, wherein the polymer matrix includes an outer surface flush with a portion of the outer surface of the electrode, which is adjacent the recess.

12. The lead of claim 8, wherein the polymer matrix includes an outer surface having a generally arcuate profile.

13. The lead of claim 8, wherein the polymer matrix includes an outer surface having a generally flat profile.

14. The lead of claim 1, wherein a ratio of the maximum diameter of the electrode outer surface to the diameter of the first and second segments of the lead body distal portion is from approximately 1.1 to approximately 1.6.

15. A medical electrode, comprising:
an active electrode surface extending from a first end to a second end and having a maximum outer diameter exceeding an outer diameter of both of the first end and the second end;
a recess formed in the active electrode surface; and
an agent held in the recess, the agent adapted to disperse out from the recess upon implantation of the electrode.

16. The electrode of claim 15, wherein the active electrode surface has a generally arcuate profile.

17. The electrode of claim 15, wherein the maximum diameter of the active electrode surface is approximately aligned with a longitudinal center of the electrode.

18. The electrode of claim 15, wherein the recess extends around a circumference of the active electrode surface.

19. The electrode of claim 15, wherein the recess is offset from a longitudinal center of the electrode.

20. The electrode of claim 15, wherein the recess is approximately aligned with a longitudinal center of the electrode.

21. The electrode of claim 15, wherein the agent is an anti-inflammatory agent.

22. The electrode of claim 15, further comprising a polymer matrix in which the agent is embedded.

23. The electrode of claim 22, wherein the polymer matrix includes an outer surface protruding from a portion of the active electrode surface, which is adjacent the recess.

24. The electrode of claim 22, wherein the polymer matrix includes an outer surface recessed below a portion of the active electrode surface, which is adjacent the recess of the active electrode surface.

25. The electrode of claim 22, wherein the polymer matrix includes an outer surface flush with a portion of the active electrode surface, which is in close proximity to the recess.

26. The electrode of claim 22, wherein the polymer matrix includes an outer surface having a generally arcuate profile.

27. The electrode of claim 22, wherein the polymer matrix includes an outer surface having a generally flat profile.

28. The electrode of claim 15, wherein a ratio of the maximum outer diameter of the active electrode surface to the outer diameter of the first end and the second end is from approximately 1.1 to approximately 1.6.
